(19) United States
(12) Patent Application Publication
(10) Pub. No.: US 2025/0078359 A1
(43) Pub. Date: Mar. 6, 2025

(54) INFORMATION PROCESSING SYSTEM, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Tokyo (JP); Kenji NAGAMIYA, Tokyo (JP); Tomohide HIRAGAMI, Tokyo (JP)

(21) Appl. No.: 18/809,356

(22) Filed: Aug. 20, 2024

(30) Foreign Application Priority Data
Sep. 1, 2023 (JP) 2023-142512

(51) Int. Cl. G06T 11/60; A61B 90/00; G06V 10/25; G06V 20/20

(52) U.S. Cl. CPC G06T 11/60; A61B 90/37; G06V 10/25; G06V 20/20; A61B 2090/365; A61B 2090/372; A61B 2090/373

(57) ABSTRACT

An information processing system including: at least one processor, wherein the processor is configured to: acquire a visual field image showing a visual field of a user; acquire, in a case where the visual field image includes a target region of interest that is a predetermined type of region of interest, relevant information related to the target region of interest; and perform control of causing a first display device viewed by the user to display the relevant information to be superimposed on the target region of interest.

### CROSS-REFERENCE TO RELATED APPLICATIONS

This application claims priority from Japanese Application No. 2023-142512, filed on Sep. 1, 2023, the entire disclosure of which is incorporated herein by reference.

### BACKGROUND

### Technical Field

The technique of the present disclosure relates to an information processing system, an information processing method, and an information processing program.

### Related Art

In the related art, a display device, such as an augmented reality (AR) device, that allows a user to visually recognize a real space and a display image is known. For example, WO2017/104666A discloses a technique of imaging a subject to acquire an imaging display image and an image for decoding, decoding the image for decoding to acquire a light ID, acquiring an AR image corresponding to the light ID and recognition information from a server, and displaying an imaging display image in which the AR image is superimposed on a region of the imaging display image in accordance with the recognition information.

In recent years, there has been a demand for a technique capable of improving visibility of at least a part of a visual field image visually recognized by a user through a display device such as a head-mounted display or smart glasses. For example, in an endoscopy, a doctor operates an endoscope while viewing an endoscopic video image until a desired observation target portion is reached. However, a display device on which the endoscopic video image is displayed may be far from the doctor depending on a standing position or the like of the doctor, and thus the endoscopic video image may be difficult to view.

### SUMMARY

The present disclosure provides an information processing system, an information processing method, and an information processing program capable of improving visibility of at least a part of a visual field.

According to a first aspect of the present disclosure, there is provided an information processing system comprising at least one processor, in which the processor is configured to acquire a visual field image showing a visual field of a user, acquire, in a case where the visual field image includes a target region of interest that is a predetermined type of region of interest, relevant information related to the target region of interest, and perform control of causing a first display device viewed by the user to display the relevant information to be superimposed on the target region of interest.

In the first aspect, the processor may be configured to acquire visual line information of the user, specify a region of interest to be paid attention that is a region of interest to which the user pays attention in the visual field image based on the visual line information, acquire, in a case where the region of interest to be paid attention is the target region of interest, the relevant information related to the target region of interest, and perform control of causing the first display device to display the relevant information to be superimposed on the target region of interest.

In the first aspect, the processor may be configured to perform control of displaying the relevant information in a region wider than the target region of interest in the visual field image.

In the first aspect, the processor may be configured to switch whether or not to display the relevant information on the first display device in response to an instruction from the user.

In the first aspect, the processor may be configured to receive a predetermined operation or utterance of the user as the instruction from the user.

In the first aspect, the target region of interest may be a region of a second display device different from the first display device, and the processor may be configured to acquire a content displayed on the second display device as the relevant information.

In the first aspect, the second display device may be a device that displays an image captured by at least one of an endoscope, a microscope, or a surgical field camera.

According to a second aspect of the present disclosure, there is provided an information processing method executed by a computer comprising acquiring a visual field image showing a visual field of a user, acquiring, in a case where the visual field image includes a target region of interest that is a predetermined type of region of interest, relevant information related to the target region of interest, and performing control of causing a first display device viewed by the user to display the relevant information to be superimposed on the target region of interest.

According to a third aspect of the present disclosure, there is provided an information processing program causing a computer to execute a process comprising acquiring a visual field image showing a visual field of a user, acquiring, in a case where the visual field image includes a target region of interest that is a predetermined type of region of interest, relevant information related to the target region of interest, and performing control of causing a first display device viewed by the user to display the relevant information to be superimposed on the target region of interest.

According to the above aspect, the information processing system, the information processing method, and the information processing program of the present disclosure can improve the visibility of at least a part of the visual field.

### BRIEF DESCRIPTION OF THE DRAWINGS

FIG. 1 is a schematic diagram showing an example of an overall configuration of an endoscope system.

FIG. 2 is a schematic diagram showing an example of the overall configuration of the endoscope system.

FIG. 3 is a schematic diagram showing an example of an overall configuration of an information processing system.

FIG. 4 is a block diagram showing an example of a hardware configuration of an HMD.

FIG. 5 is a block diagram showing an example of a hardware configuration of an information processing apparatus.

FIG. 6 is a block diagram showing an example of a functional configuration of the information processing apparatus.

FIG. 7 is a diagram showing an example of a visual field image.

FIG. 8 is a diagram showing an example of an image displayed on the HMD.

FIG. 9 is a flowchart showing an example of information processing.

FIG. 10 is a diagram showing an example of a visual field image.

FIG. 11 is a diagram showing an example of the image displayed on an HMD.

### DETAILED DESCRIPTION

Hereinafter, an embodiment of the present disclosure will be described with reference to accompanying drawings.

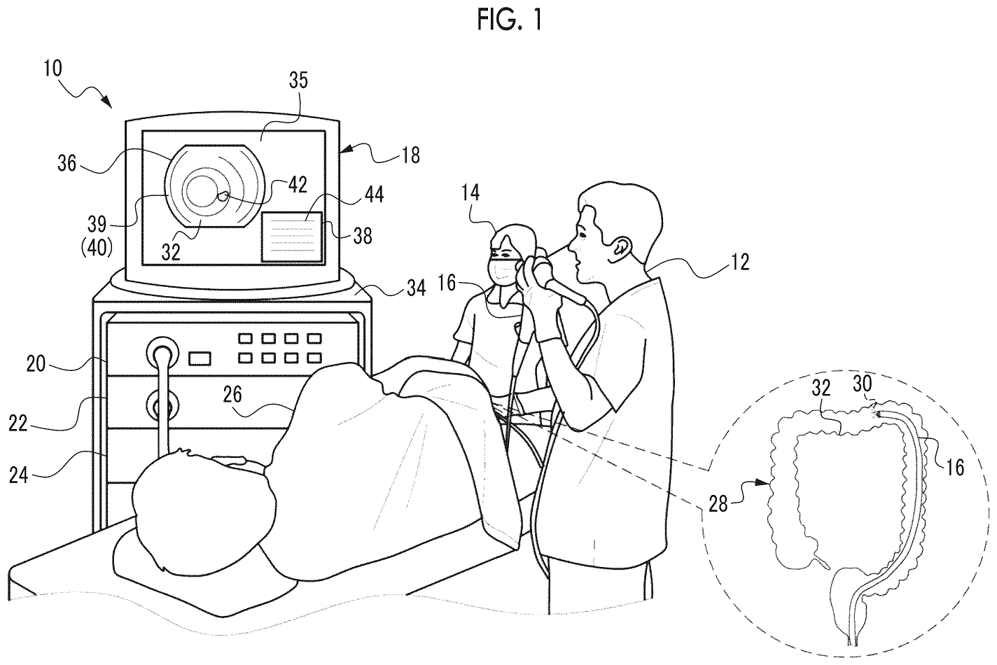

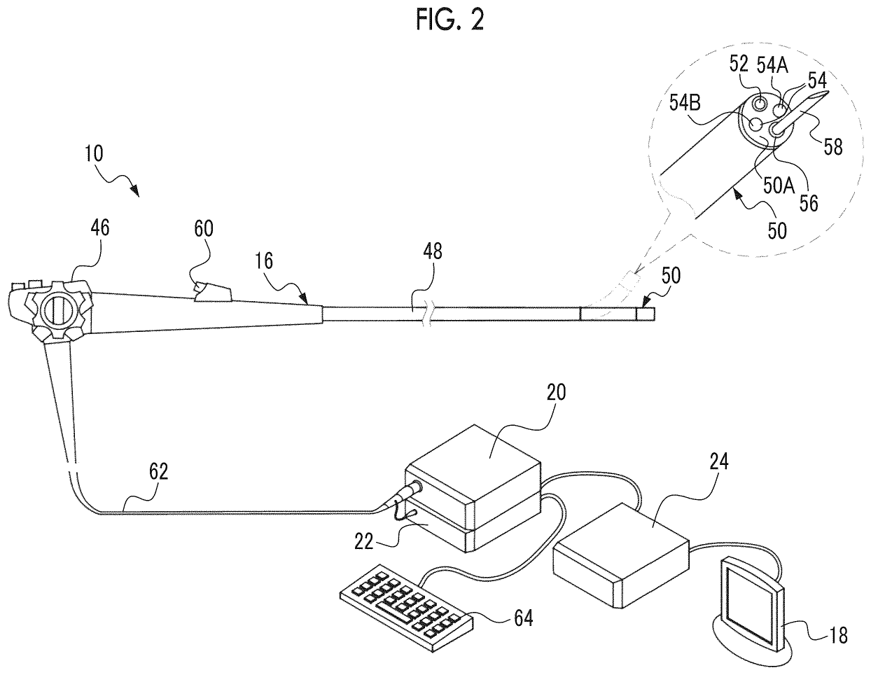

First, an endoscope system 10 to which the technique of the present disclosure can be applied will be described with reference to FIGS. 1 and 2. The endoscope system 10 is used by a doctor 12 in endoscopy and the like. The endoscopy is assisted by a staff such as a nurse 14.

As shown in FIG. 1, an endoscope system 10 comprises an endoscope 16, a display device 18, a light source device 20, a control device 22, and an image processing device 24. The light source device 20, the control device 22, and the image processing device 24 are installed in a wagon 34. A plurality of tables are provided in the wagon 34 along a vertical direction, and the image processing device 24, the control device 22, and the light source device 20 are installed from a lower table to an upper table. Further, the display device 18 is installed on an uppermost table in the wagon 34.

The endoscope system 10 is a modality for performing medical treatment on the inside of a body of a subject 26 (for example, a patient) using the endoscope 16. The endoscope 16 is used by the doctor 12 and is inserted into a body cavity (for example, luminal organs such as large intestine, stomach, duodenum, and trachea) of the subject 26. The endoscope system 10 causes the endoscope 16 inserted into the body cavity of the subject 26 to image the inside of the body cavity of the subject 26 and performs various medical treatments in the body cavity as necessary.

For example, in the present embodiment, the endoscope 16 is inserted into a large intestine 28 of the subject 26. The endoscope system 10 images the inside of the large intestine 28 of the subject 26 to acquire and output an image showing an aspect inside the large intestine 28. In the present embodiment, the endoscope system 10 has an optical imaging function of emitting light 30 in the large intestine 28 to image reflected light obtained by being reflected by an intestinal wall 32 of the large intestine 28.

The control device 22 controls the entire endoscope system 10. The image processing device 24 performs various types of image processing on the image obtained by imaging the intestinal wall 32 with the endoscope 16 under the control of the control device 22.

The display device 18 displays various types of information including the image. Examples of the display device 18 include a liquid crystal display and an organic electro-luminescence (EL) display. Further, a smartphone, a tablet terminal, or the like may be used instead of or in addition to the display device 18. The display device 18 is an example of a second display device of the present disclosure.

A screen 35 is displayed on the display device 18. The screen 35 includes a plurality of display regions. The plurality of display regions are disposed side by side on the screen 35. In the example shown in FIG. 1, a first display region 36 and a second display region 38 are shown as an example of the plurality of display regions. The first display region 36 is used as a main display region, and the second display region 38 is used as a sub-display region.

An endoscopic video image 39 is displayed in the first display region 36. The endoscopic video image 39 is a video image acquired by imaging the intestinal wall 32 in the large intestine 28 of the subject 26 with the endoscope 16. The example shown in FIG. 1 shows a video image in which the intestinal wall 32 appears, as an example of the endoscopic video image 39.

In the example in FIG. 1, the intestinal wall 32 appearing in the endoscopic video image 39 includes a lesion 42 as an observation target region gazed by the doctor 12. The doctor 12 can visually recognize the aspect of the intestinal wall 32 including the lesion 42 through the endoscopic video image 39. Examples of the lesion 42 include a tumor-like polyp and a non-tumor-like polyp.

The image displayed in the first display region 36 is one frame 40, which is included in the video image configured by including a plurality of frames 40 in time series. That is, the plurality of frames 40 in time series are displayed at a predetermined frame rate (for example, several tens of frames/second) in the first display region 36.

Medical information 44, which is information related to medical treatment, is displayed in the second display region 38. Examples of the medical information 44 include information for assisting medical determination or the like by the doctor 12.

Examples of such information include various types of information related to the subject 26 and information obtained by performing image analysis using a computer aided diagnosis/detection (CAD) technique on the endoscopic video image 39.

As shown in FIG. 2, the endoscope 16 comprises an operation part 46 and an insertion part 48. The insertion part 48 is partially curved by the operation of the operation part 46. The insertion part 48 is inserted into the large intestine 28 while being bent in accordance with a shape of the large intestine 28 in response to the operation of the operation part 46 by the doctor 12.

A tip part 50 of the insertion part 48 is provided with a camera 52, an illumination device 54, and an opening for treatment tool 56. The camera 52 and the illumination device 54 are provided on a distal end surface 50A of the tip part 50. Here, although the form example has been described in which the camera 52 and the illumination device 54 are provided on the distal end surface 50A of the tip part 50, the present disclosure is not limited thereto. The camera 52 and the illumination device 54 may be provided on a side surface of the tip part 50, and thus the endoscope 16 may be configured as a side-viewing endoscope.

The camera 52 is inserted into the body cavity of the subject 26 to image the observation target region. In the present embodiment, the camera 52 images the inside of the body of the subject 26 (for example, the inside of the large intestine 28) to acquire the endoscopic video image 39. Examples of the camera 52 include a complementary metal oxide semiconductor (CMOS) camera and a charge coupled device (CCD) camera.

The illumination device 54 has illumination windows 54A and 54B. The illumination device 54 emits the light 30 via the illumination windows 54A and 54B. Examples of a type of the light 30 emitted from the illumination device 54 include visible light (for example, white light) and invisible light (for example, near-infrared light).

Further, the illumination device 54 emits special light via the illumination windows 54A and 54B. Examples of the special light include light for blue light imaging (BLI) and light for linked color imaging (LCI). The camera 52 images the inside of the large intestine 28 by an optical method in a state where the illumination device 54 emits the light 30 in the large intestine 28.

The opening for treatment tool 56 is an opening through which a treatment tool 58 protrudes from the tip part 50. Further, the opening for treatment tool 56 is also used as a suction port that sucks blood, a body waste, and the like, and as a discharge port that discharges a fluid.

A treatment tool insertion port 60 is formed in the operation part 46, and the treatment tool 58 is inserted into the insertion part 48 from the treatment tool insertion port 60. The treatment tool 58 passes through the insertion part 48 and protrudes from the opening for treatment tool 56 to the outside. The example shown in FIG. 2 shows an aspect in which a puncture needle protrudes from the opening for treatment tool 56, as the treatment tool 58. The treatment tool 58 is not limited to the puncture needle, and may be, for example, a grasping forceps, a papillotomy knife, a snare, a catheter, a guide wire, a cannula, or a puncture needle with a guide sheath.

The endoscope 16 is connected to the light source device 20 and the control device 22 via a universal cord 62. The image processing device 24 and a reception device 64 are connected to the control device 22. Further, the display device 18 is connected to the image processing device 24. That is, the control device 22 is connected to the display device 18 via the image processing device 24.

The reception device 64 receives an instruction from the doctor 12 and outputs the received instruction as an electric signal to the control device 22. Examples of the reception device 64 include a keyboard, a mouse, a touch panel, a foot switch, a microphone, and a remote control device.

The control device 22 controls the light source device 20, exchanges various signals with the camera 52, or exchanges various signals with the image processing device 24.

The light source device 20 emits light under the control of the control device 22 and supplies the light to the illumination device 54. The illumination device 54 has a built-in light guide, and the light supplied from the light source device 20 is emitted from the illumination windows 54A and 54B through the light guide. The control device 22 causes the camera 52 to perform imaging, acquires the endoscopic video image 39 from the camera 52, and outputs the endoscopic video image 39 to a predetermined output destination (for example, the image processing device 24).

The image processing device 24 performs various types of image processing on the endoscopic video image 39 input from the control device 22 to support the endoscopy. The image processing device 24 outputs the endoscopic video image 39 subjected to various types of image processing to a predetermined output destination (for example, the display device 18 and the control device 22).

Here, since the image processing device 24 is exemplified as an external device for extending the function performed by the control device 22, the form example is exemplified in which the control device 22 and the display device 18 are indirectly connected via the image processing device 24, but the present disclosure is not limited thereto. For example, the display device 18 may be directly connected to the control device 22. In this case, for example, the control device 22 may be provided with the function of the image processing device 24, or the control device 22 may be provided with a function of executing the same processing as the processing executed by the image processing device 24 on a server (not illustrated) and receiving and using a processing result by the server.

Further, the endoscope system 10 is communicably connected to an information processing apparatus 300 and another external device (not illustrated). Examples of the external device include a server and/or a client terminal (for example, a personal computer, a smartphone, and a tablet terminal) that manage various types of information such as an electronic medical record. The external device receives the information transmitted from the endoscope system 10 and executes processing using the received information (for example, processing of storing the information in the electronic medical record or the like).

However, in the endoscope system 10, the doctor 12 operates the operation part 46 with the left hand and operates the insertion part 48 with the right hand while viewing the display device 18 such that the tip part 50 of the endoscope 16 reaches a desired observation target region. In particular, in a case where the large intestine 28 is to be examined, since there is a large individual difference in the movement of the large intestine 28, it is necessary to flexibly change the operation in accordance with the state of the subject 26 (for example, a calm state, a distressed state, or the like).

In order to perform such a complicated operation, all of the display device 18, the operation part 46 of the left hand, the insertion part 48 of the right hand, and the subject 26 are required to be included in the visual field. However, in that case, a distance from the doctor to the display device 18 may be long, and the visibility of the endoscopic video image 39 may be decreased. The information processing system 100 of the present disclosure improves the visibility of at least a part of the visual field in the display device viewed by the user.

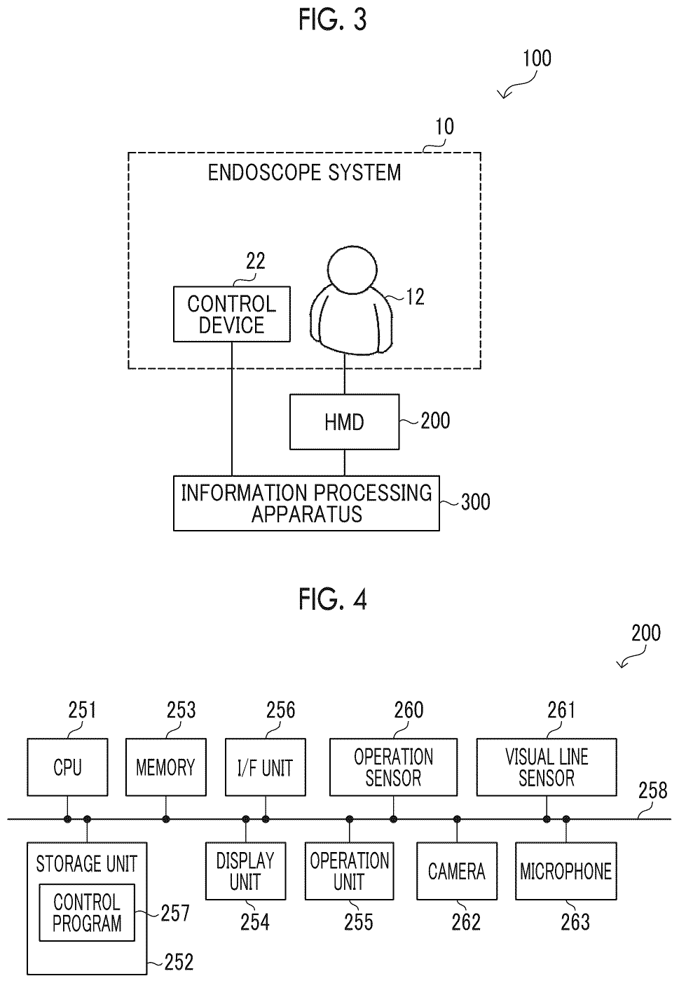

The information processing system 100 of the present disclosure will be described with reference to FIG. 3. In the present embodiment, the information processing system 100 includes the information processing apparatus 300 and the endoscope system 10 used by the doctor 12. Further, in the information processing system 100, the doctor 12 wears a head mounted display (HMD) 200 as an example of a first display device viewed by the user.

An example of a hardware configuration of the HMD 200 will be described with reference to FIG. 4. As shown in FIG. 4, the HMD 200 includes a central processing unit (CPU) 251, a non-volatile storage unit 252, and a memory 253 as a temporary storage region. Further, the HMD 200 includes a display unit 254, an operation unit 255 such as a touch panel, and an interface (I/F) unit 256. Further, the HMD 200 includes an operation sensor 260 that detects an operation of a user to whom the HMD 200 is mounted, a visual line sensor 261 that detects a visual line, a camera 262, and a microphone 263.

The storage unit 252 is formed by, for example, a storage medium such as a flash memory. The storage unit 252 stores a control program 257 that controls the entire HMD 200. The CPU 251 reads out the control program 257 from the storage unit 252, develops the control program 257 into the memory 253, and executes the developed control program 257.

The display unit 254 is configured such that the user wearing the HMD 200 can visually recognize an image (virtual image). For example, the display unit 254 may use a transmissive or non-transmissive display, or may project a video to a retina of the user. The I/F unit 256 performs wired or wireless communication with the information processing apparatus 300, another external device, and the like.

The operation sensor 260 is a sensor that detects the operation of the user wearing the HMD 200, and is, for example, an acceleration sensor, a gyro sensor, a geomagnetic sensor, or the like. The visual line sensor 261 is a sensor that detects where a gaze point of the user wearing the HMD 200 is. The camera 262 is a camera that images a real space observed by the user wearing the HMD 200, and is, for example, a digital camera such as a CMOS camera. The microphone 263 collects a voice and an ambient sound of the user wearing the HMD 200.

The CPU 251, the storage unit 252, the memory 253, the display unit 254, the operation unit 255, the I/F unit 256, the operation sensor 260, the visual line sensor 261, the camera 262, and the microphone 263 are connected to be able to mutually exchange various types of information via a bus 258, such as a system bus and a control bus.

The information processing apparatus 300 is configured to be connectable to the control device 22, the HMD 200, and another external device (not shown) of the endoscope system 10, respectively, through a wired or wireless network.

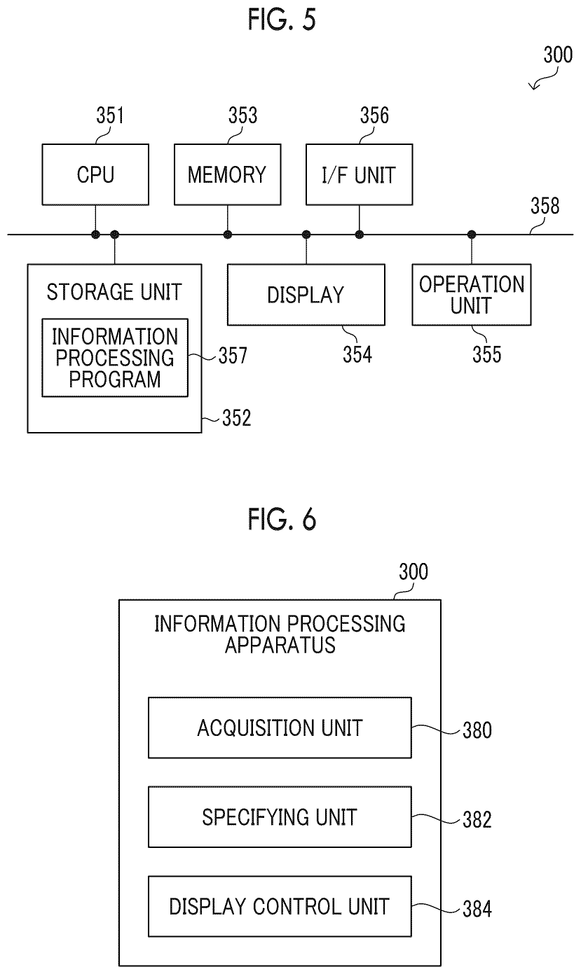

An example of a hardware configuration of the information processing apparatus 300 will be described with reference to FIG. 5. As shown in FIG. 5, the information processing apparatus 300 includes a CPU 351, a non-volatile storage unit 352, and a memory 353 as a temporary storage region. Further, the information processing apparatus 300 includes a display 354, such as a liquid crystal display, an operation unit 355, such as a touch panel, a keyboard, and a mouse, and an I/F unit 356. The I/F unit 356 performs wired or wireless communication with the control device 22, the HMD 200, an external device, and the like.

The storage unit 352 is formed by a storage medium such as a hard disk drive (HDD), a solid state drive (SSD), and a flash memory. The storage unit 352 stores an information processing program 357 in the information processing apparatus 300. The CPU 351 reads out the information processing program 357 from the storage unit 352, develops the information processing program 357 into the memory 353, and executes the developed information processing program 357.

The CPU 351, the storage unit 352, the memory 353, the display 354, the operation unit 355, and the I/F unit 356 are connected to be able to mutually exchange various types of information via a bus 358, such as a system bus and a control bus. As the information processing apparatus 300, for example, a personal computer, a server computer, a smartphone, a tablet terminal, a wearable terminal, or the like can be applied as appropriate.

An example of a functional configuration of the information processing apparatus 300 will be described with reference to FIG. 6. As shown in FIG. 6, the information processing apparatus 300 includes an acquisition unit 380, a specifying unit 382, and a display control unit 384. With the execution of the information processing program 357 by the CPU 351, the CPU 351 functions as the acquisition unit 380, the specifying unit 382, and the display control unit 384.

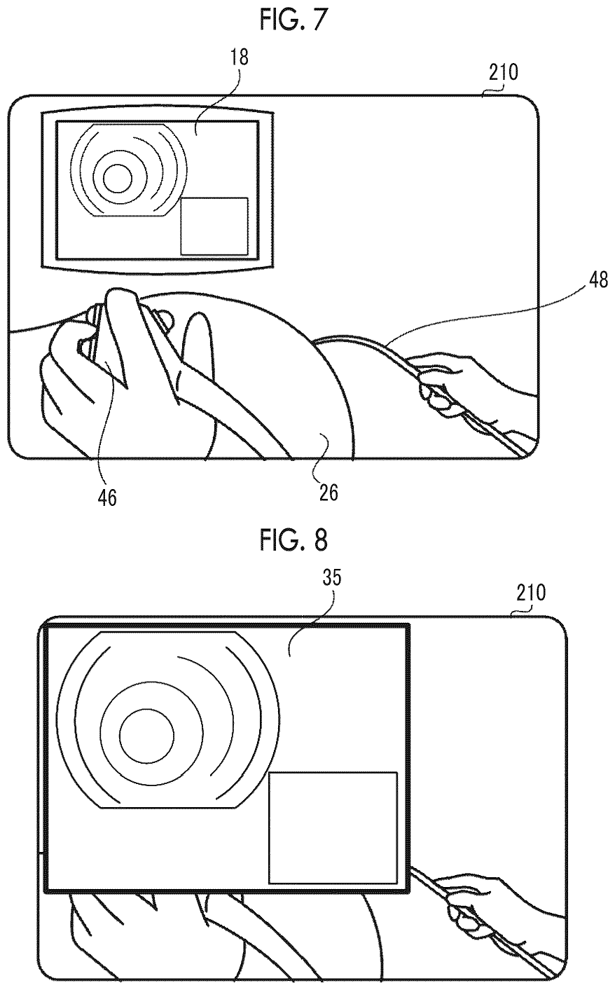

The acquisition unit 380 acquires a visual field image 210 indicating the visual field of the doctor 12 from the HMD 200 worn by the doctor 12. As an example, FIG. 7 shows the visual field image 210 showing the visual field of the doctor 12 who operates the endoscope system 10. In general, in the endoscopy, model states such as the holding method of the endoscope 16, a standing position with respect to the subject 26 and the display device 18, and the visual field (an object within field of view and a position thereof) are predetermined. The visual field image 210 in FIG. 7 is an appropriate state in which all of the display device 18, the operation part 46 held in the left hand, the insertion part 48 held in the right hand, and the state of the subject 26 (insertion port) can be visually recognized at the same time. However, as a result, a distance from a viewpoint of the doctor to the display device 18 is long, and the visibility of the display device 18 is decreased. The doctor 12 is an example of the user according to the present disclosure.

In a case where the visual field image 210 acquired by the acquisition unit 380 includes a target region of interest, which is a predetermined type of region of interest, the specifying unit 382 acquires relevant information related to the target region of interest. In other words, the target region of interest is a region where the relevant information is associated in advance and the relevant information can be acquired.

Examples of such a target region of interest include a region of various display devices. In a case where the visual field image 210 includes the region of the display device 18, the specifying unit 382 may acquire a content displayed by the display device 18 as the relevant information. The display device 18 is not limited to the endoscope as shown in FIG. 1, and may be, for example, a device that displays an image captured by at least one of an endoscope, a microscope, or a surgical field camera.

For example, in the example of FIG. 7, since the visual field image 210 of the doctor 12 includes the region of the display device 18, which is the target region of interest, the specifying unit 382 acquires the relevant information related to the display device 18. Specifically, the specifying unit 382 acquires, as the relevant information, information (including the endoscopic video image 39 and the medical information 44) of the screen 35 displayed on the display device 18 by the endoscope system 10, from the control device 22 of the endoscope system 10.

The display control unit 384 performs control of causing the HMD 200 worn by the doctor 12 to display the relevant information acquired by the specifying unit 382 to be superimposed on the target region of interest in the visual field image 210. As an example, FIG. 8 is a diagram in which the screen 35 displayed on the display device 18 by the endoscope system 10 is enlarged and displayed in a superimposed manner on the region (target region of interest) of the display device 18 in the visual field image 210. As described above, the display control unit 384 may control such that the relevant information is displayed in the region wider than the target region of interest in the visual field image 210. That is, the display control unit 384 may enlarge and display the relevant information.

Further, in a case where the relevant information is displayed in a superimposed manner only by being included in the visual field, it may be rather troublesome. The relevant information may be displayed in a superimposed manner only in a case where the doctor 12 pays attention to the target region of interest. Specifically, the acquisition unit 380 may acquire visual line information of the doctor 12, which is detected by the visual line sensor 261 provided in the HMD 200 of the doctor 12.

The specifying unit 382 may specify a region of interest to be paid attention, which is the region of interest to which the doctor 12 pays attention in the visual field image 210, based on the visual line information acquired by the acquisition unit 380. For example, it is assumed that the visual line information of the doctor 12 indicates that the display device 18 is paid attention. In this case, the specifying unit 382 specifies that the display device 18 is the region of interest to be paid attention among respective regions of interest (the display device 18, the operation part 46, the insertion part 48, the subject 26, and the like) included in the visual field image 210.

Further, in a case where the specified region of interest to be paid attention is also the target region of interest (for example, the display device or the like), the specifying unit 382 may acquire the relevant information related to the target region of interest. For example, in a case where the region of interest to be paid attention is the display device 18, the region of interest to be paid attention corresponds to the target region of interest. Therefore, the specifying unit 382 acquires the information of the screen 35 displayed on the display device 18 as the relevant information from the control device 22 of the endoscope system 10.

Further, the doctor 12 may be able to check the relevant information or stop the check at any timing. Specifically, the display control unit 384 may switch whether or not to display the relevant information on the HMD 200 in response to the instruction from the doctor 12.

The display control unit 384 may receive a predetermined operation or utterance of the doctor 12 as the instruction from the doctor 12. For example, in a case where the operation sensor 260 provided in the HMD 200 detects that the doctor 12 performs a predetermined operation (for example, nodding twice), the display control unit 384 may receive the instruction. Further, for example, in a case where a region for instruction is provided in an end part or the like of the HMD 200 and the visual line sensor 261 detects that the visual line is directed to the region for instruction for a predetermined time or longer, the instruction may be received. Further, for example, the instruction by a voice input from the doctor 12 may be received by using the microphone 263 provided in the HMD 200.

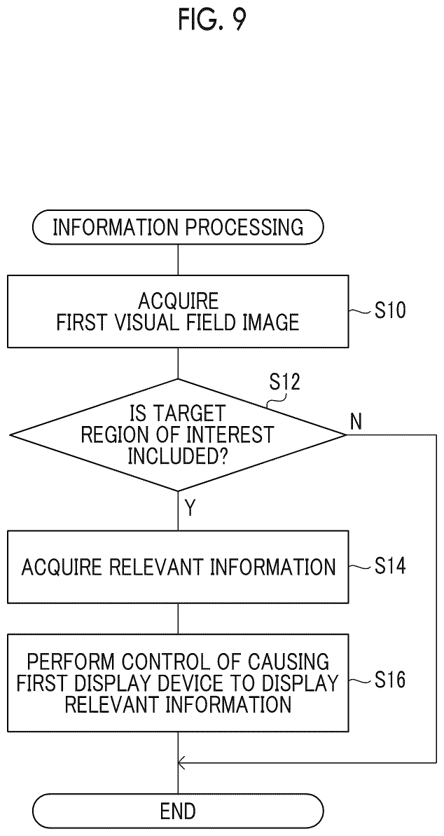

Next, an action of the information processing apparatus 300 according to the present embodiment will be described with reference to FIG. 9. In the information processing apparatus 300, with the execution of the information processing program 357 by the CPU 351, information processing shown in FIG. 9 is executed. The information processing is executed, for example, in a case where the user gives an instruction to start execution via the operation unit 355.

In step S10, the acquisition unit 380 acquires the visual field image 210 showing the visual field of the user (doctor 12). In step S12, the specifying unit 382 determines whether or not the visual field image 210 acquired in step S10 includes the target region of interest, which is the predetermined type of region of interest.

In a case where the target region of interest is included, the processing proceeds to step S14, and the specifying unit 382 acquires the relevant information related to the target region of interest. In step S16, the display control unit 384 performs control of causing the first display device (HMD 200) viewed by the user (doctor 12) to display the relevant information acquired in step S14 to be superimposed on the target region of interest in the visual field image 210 acquired in step S10. In a case where the target region of interest is not included in step S12 and in a case where step S16 is completed, the present information processing ends.

As described above, the information processing system 100 according to one aspect of the present disclosure comprises at least one processor, and the processor acquires the visual field image 210 showing the visual field of the user (doctor 12). Further, in a case where the visual field image 210 includes the target region of interest, which is the predetermined type of region of interest, the relevant information related to the target region of interest is acquired. Further, the first display device (HMD 200) viewed by the user (doctor 12) is controlled to display the relevant information to be superimposed on the target region of interest.

That is, with the information processing system 100 of the present disclosure, since the relevant information can be displayed for the target region of interest, the visibility of at least a part of the visual field can be improved.

In the above embodiment, the form has been described in which the target region of interest is the second display device (display device 18) and the relevant information is the display content of the second display device. However, the present disclosure is not limited thereto.

For example, the operation part 46 or the like of the endoscope 16 may be applied as the target region of interest. In this case, for example, parameter information of the endoscope 16 can be applied as the relevant information. Further, for example, an image obtained by capturing the operation part 46 using a camera other than the camera (camera 262 of the HMD 200) that captures the visual field image 210 may be applied as the relevant information. For example, an image captured by a camera that images the operation part 46 from a side surface may be acquired.

Further, in the above embodiment, the form has been described in which the technique of the present disclosure is applied to the endoscope system 10, but the present disclosure is not limited thereto. The technique of the present disclosure can be applied to a medical field and various other fields.

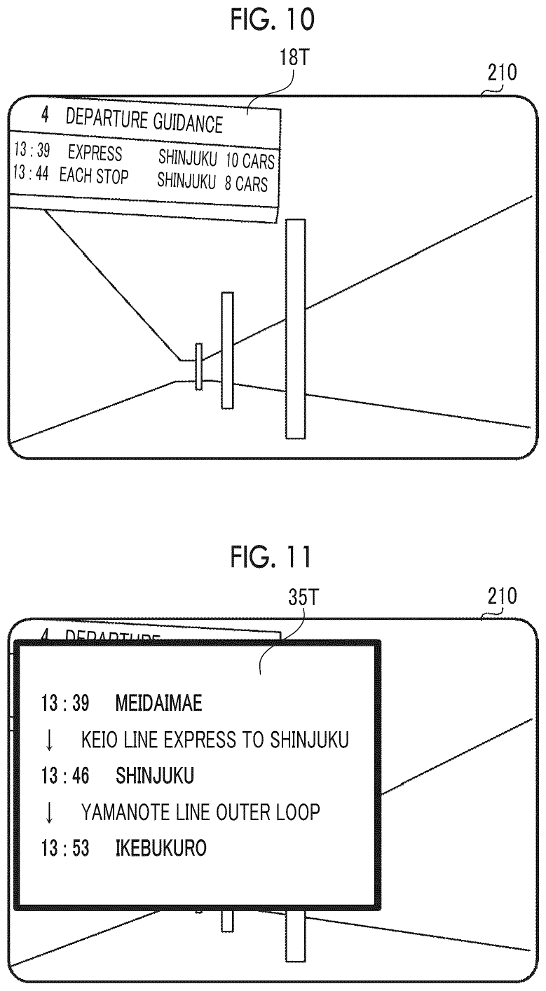

As an example, FIGS. 10 and 11 show an example in which the target region of interest is a departure guidance board 18T of a railway passenger station and the relevant information is a transfer guidance 35T. As shown in FIG. 10, in a case where a departure time point of a train is to be known at a platform of a station, poor visibility is obtained in a case where a distance from the departure guidance board 18T is long. In such a case, with application of the technique of the present disclosure, the departure time point of the train can be visually recognized without approaching the departure guidance board 18T.

Further, as shown in FIG. 11, the transfer guidance may be displayed as the relevant information. A destination may be set to, for example, a predetermined destination such as a nearest station to a home, a workplace, and a school, or may be input by the user each time. A content of the departure guidance board 18T and information of the transfer guidance may be acquired, for example, from a website that provides the transfer guidance, a timetable, and the like by using an application programming interface (API) with a departure point and/or the destination as a search query.

Further, in the above embodiment, the form has been described in which the visual field image 210 is captured by the camera 262 provided in the HMD 200, but the present disclosure is not limited thereto. For example, the visual field image 210 may be acquired by using a camera provided at any place such as a wall and a ceiling of a room where the user is present and a camera mounted at any part of the head, the shoulder, the chest, and the like of the user. Further, for example, these external cameras and the camera 262 provided in the HMD 200 may be combined to generate the visual field image in a wider range. Further, for example, the image displayed on the display unit 254 of the HMD 200 may be captured by using the camera 262 of the HMD 200, and the image used for extracting the target region of interest may be captured by using an external camera.

Further, in the above embodiment, the form example has been described in which the HMD 200 is applied as the first display device viewed by the user, but the present disclosure is not limited thereto. As the first display device viewed by the user of the present disclosure, for example, various portable displays such as a glasses-type wearable device (so-called smart glasses) and a contact lens-type wearable device (so-called smart contact lens) may be applied. Further, for example, a stationary display disposed near each user may be applied. Further, for example, a screen that is suspended from a ceiling of a room where each user stays or a projector that projects an image to a wall of the room may be applied. Further, for example, AR glasses that are suspended from a ceiling of a room where each user stays may be applied, and each user may look into the AR glasses. Further, for example, an aerial display that forms an image in the air may be applied.

Further, in the above embodiment, the control device 22 or the like of the endoscope system 10 may include a part or all of the functions of the information processing apparatus 300, such as the acquisition unit 380, the specifying unit 382, and the display control unit 384.

In the above embodiment, for example, as hardware structures of processing units that execute various types of processing, such as the acquisition unit 380, the specifying unit 382, and the display control unit 384, various processors shown below can be used. As described above, in addition to the CPU that is a general-purpose processor that executes software (program) to function as various processing units, the various processors include a programmable logic device (PLD) that is a processor of which a circuit configuration can be changed after manufacture, such as a field programmable gate array (FPGA), and a dedicated electric circuit that is a processor having a circuit configuration that is designed for exclusive use in order to execute a specific process, such as an application specific integrated circuit (ASIC).

One processing unit may be configured by using one of the various processors or may be configured by using a combination of two or more processors of the same type or different types (for example, a combination of a plurality of FPGAs or a combination of a CPU and an FPGA). Moreover, a plurality of processing units may be configured of one processor.

As an example of configuring the plurality of processing units with one processor, first, there is a form in which one processor is configured by a combination of one or more CPUs and software and the processor functions as the plurality of processing units, as represented by computers such as a client and a server. Second, as represented by a system-on-chip (SoC) or the like, there is a form in which the processor is used in which the functions of the entire system which includes the plurality of processing units are realized by a single integrated circuit (IC) chip. In this manner, the various processing units are configured using one or more of the various processors as a hardware structure.

Further, more specifically, a circuitry combining circuit elements such as semiconductor elements can be used as the hardware structure of the various processors.

Further, in the above embodiment, the form has been described in which the various programs in the information processing apparatus 300 and the HMD 200 are stored in the storage unit in advance, but the present disclosure is not limited thereto. The various programs may be provided in a form of being recorded on a recording medium such as a compact disc read only memory (CD-ROM), a digital versatile disc read only memory (DVD-ROM), and a Universal Serial Bus (USB) memory. The various programs may be provided in a form of being downloaded from an external device via a network. Furthermore, the technique of the present disclosure extends to a storage medium that stores the program non-transitorily, in addition to the program.

In the technique of the present disclosure, the embodiment and the examples described above can be combined as appropriate. The above-described contents and the above-shown contents are detailed descriptions for parts according to the technique of the present disclosure, and are merely examples of the technique of the present disclosure. For example, the descriptions regarding the configurations, the functions, the actions, and the effects are descriptions regarding an example of the configurations, the functions, the actions, and the effects of the part according to the technique of the present disclosure. Accordingly, in the contents described and the contents shown hereinabove, it is needless to say that removal of an unnecessary part, or addition or replacement of a new element may be employed within a range not departing from the gist of the technique of the present disclosure.

Regarding the above embodiment, the following Supplementary Notes are further disclosed.

### Supplementary Note 1

An information processing system comprising:
at least one processor,
wherein the processor is configured to:
acquire a visual field image showing a visual field of a user;
acquire, in a case where the visual field image includes a target region of interest that is a predetermined type of region of interest, relevant information related to the target region of interest; and
perform control of causing a first display device viewed by the user to display the relevant information to be superimposed on the target region of interest.

### Supplementary Note 2

The information processing system according to Supplementary Note 1,
wherein the processor is configured to:
acquire visual line information of the user;
specify a region of interest to be paid attention that is a region of interest to which the user pays attention in the visual field image based on the visual line information;
acquire, in a case where the region of interest to be paid attention is the target region of interest, the relevant information related to the target region of interest; and
perform control of causing the first display device to display the relevant information to be superimposed on the target region of interest.

### Supplementary Note 3

The information processing system according to Supplementary Note 1 or 2,
wherein the processor is configured to:
perform control of displaying the relevant information in a region wider than the target region of interest in the visual field image.

### Supplementary Note 4

The information processing system according to any one of Supplementary Notes 1 to 3,
wherein the processor is configured to:
switch whether or not to display the relevant information on the first display device in response to an instruction from the user.

### Supplementary Note 5

The information processing system according to Supplementary Note 4,
wherein the processor is configured to:
receive a predetermined operation or utterance of the user as the instruction from the user.

### Supplementary Note 6

The information processing system according to any one of Supplementary Notes 1 to 5,
wherein the target region of interest is a region of a second display device different from the first display device, and
the processor is configured to:
acquire a content displayed on the second display device as the relevant information.

### Supplementary Note 7

The information processing system according to Supplementary Note 6,
wherein the second display device is a device that displays an image captured by at least one of an endoscope, a microscope, or a surgical field camera.

### Supplementary Note 8

An information processing method executed by a computer, the information processing method comprising:
acquiring a visual field image showing a visual field of a user;
acquiring, in a case where the visual field image includes a target region of interest that is a predetermined type of region of interest, relevant information related to the target region of interest; and
performing control of causing a first display device viewed by the user to display the relevant information to be superimposed on the target region of interest.

### Supplementary Note 9

An information processing program causing a computer to execute a process comprising:
acquiring a visual field image showing a visual field of a user;
acquiring, in a case where the visual field image includes a target region of interest that is a predetermined type of region of interest, relevant information related to the target region of interest; and
performing control of causing a first display device viewed by the user to display the relevant information to be superimposed on the target region of interest.

What is claimed is:

1. An information processing system comprising: at least one processor, wherein the processor is configured to:
acquire a visual field image showing a visual field of a user;
acquire, in a case where the visual field image includes a target region of interest that is a predetermined type of region of interest, relevant information related to the target region of interest; and
perform control of causing a first display device viewed by the user to display the relevant information to be superimposed on the target region of interest.

2. The information processing system according to claim 1, wherein the processor is configured to:
acquire visual line information of the user;
specify a region of interest to be paid attention that is a region of interest to which the user pays attention in the visual field image based on the visual line information;
acquire, in a case where the region of interest to be paid attention is the target region of interest, the relevant information related to the target region of interest; and
perform control of causing the first display device to display the relevant information to be superimposed on the target region of interest.

3. The information processing system according to claim 1, wherein the processor is configured to perform control of displaying the relevant information in a region wider than the target region of interest in the visual field image.

4. The information processing system according to claim 1, wherein the processor is configured to switch whether or not to display the relevant information on the first display device in response to an instruction from the user.

5. The information processing system according to claim 4, wherein the processor is configured to receive a predetermined operation or utterance of the user as the instruction from the user.

6. The information processing system according to claim 1, wherein:
the target region of interest is a region of a second display device different from the first display device, and
the processor is configured to acquire a content displayed on the second display device as the relevant information.

7. The information processing system according to claim 6, wherein the second display device is a device that displays an image captured by at least one of an endoscope, a microscope, or a surgical field camera.

8. An information processing method executed by a computer, the information processing method comprising:
acquiring a visual field image showing a visual field of a user;
acquiring, in a case where the visual field image includes a target region of interest that is a predetermined type of region of interest, relevant information related to the target region of interest; and
performing control of causing a first display device viewed by the user to display the relevant information to be superimposed on the target region of interest.

9. A non-transitory computer-readable storage medium storing an information processing program causing a computer to execute a process comprising:
acquiring a visual field image showing a visual field of a user;
acquiring, in a case where the visual field image includes a target region of interest that is a predetermined type of region of interest, relevant information related to the target region of interest; and
performing control of causing a first display device viewed by the user to display the relevant information to be superimposed on the target region of interest.
